(19) United States
(12) Patent Application Publication
(10) Pub. No.: US 2017/0303819 A1
(43) Pub. Date: Oct. 26, 2017

(54) METHOD AND SYSTEM FOR ALCOHOL SENSING DEVICE CHARACTERIZATION

(71) Applicant: KHN Solutions, Inc., San Francisco, CA (US)

(72) Inventors: Keith Harry Nothacker, San Francisco, CA (US); Will Tammen, San Francisco, CA (US)

(21) Appl. No.: 15/492,216

(22) Filed: Apr. 20, 2017

Related U.S. Application Data
(60) Provisional application No. 62/336,259, filed on May 13, 2016.
(60) Provisional application No. 62/325,556, filed on Apr. 21, 2016.

(51) Int. Cl. A61B 5/08; G08B 21/18; G01N 33/98

(52) U.S. Cl. CPC A61B 5/082; G01N 33/98; G08B 21/18; A61B 2560/0252; A61B 2560/0247; A61B 2560/0271

(57) ABSTRACT

A method for characterizing the state of an alcohol sensor comprising prompting an individual to provide a biological sample at a first time point; generating an alcohol signal upon reception of the biological sample from the individual; an environmental metric associated with the fuel-cell alcohol sensing device at a second time point contemporaneous with the first time point; determining a degeneration parameter of the fuel-cell alcohol sensing device; extracting a correction factor upon implementing a rule with the environmental metric and the degeneration parameter; and at the remote computing system, generating a notification based upon the correction factor exceeding a threshold correction factor.

### CROSS REFERENCE TO RELATED APPLICATIONS

This application claims the benefit of U.S. Provisional Application No. 62/336,259, filed 13-May-2016, which is incorporated in its entirety by this reference. This application also claims the benefit of U.S. Provisional Application No. 62/325,556, filed 21-Apr.-2016, which is incorporated in its entirety by this reference.

### TECHNICAL FIELD

This invention relates generally to the intoxication monitoring device field, and more specifically to a new and useful method and system for alcohol sensing device characterization.

### BACKGROUND

It is often desirable to analyze a biological sample from a person to detect substances carried in the biological sample. As such, alcohol sensing devices are used to test the content of alcohol (i.e., ethanol) carried in an individual's breath, in order to determine a measure of alcohol consumed by the individual. The measure is typically presented as a blood alcohol content (BAC), which can provide an indication of a user's mental and/or physical adeptness resulting from intoxication. As such, BAC measures are also used to provide a basis for limits of alcohol consumption in relation to the performance of tasks, including driving a vehicle, operating machinery, and performing various tasks in a working environment. While current blood alcohol measuring devices are able to determine an individual's BAC, and are typically used in law enforcement settings, existing systems and methods configured to ensure that such devices are performing properly in a consumer use setting are significantly deficient in many ways.

There is thus a need in the intoxication monitoring device field to create a new and useful method and system for alcohol sensing device characterization. This invention provides such a new and useful method and system.

### BRIEF DESCRIPTION OF THE FIGURES

FIG. 1A-C depict a flow charts of embodiments of a method for alcohol sensing device characterization;

FIG. 2 depicts example response curves in an embodiment of a system and method for alcohol sensing device characterization;

FIGS. 3A and 3B depict embodiments of systems for alcohol sensing device characterization; and

FIG. 4 depicts a portion of a variation of a system for alcohol sensing device characterization.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Method

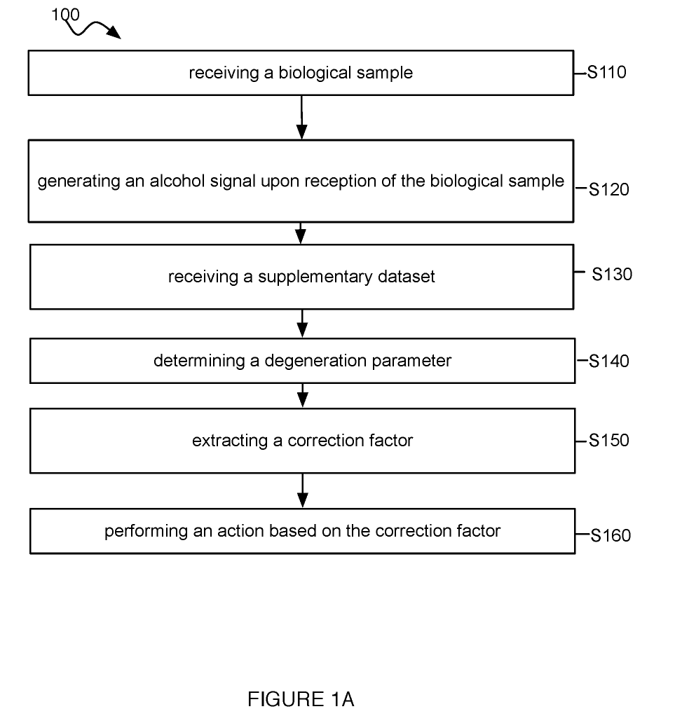

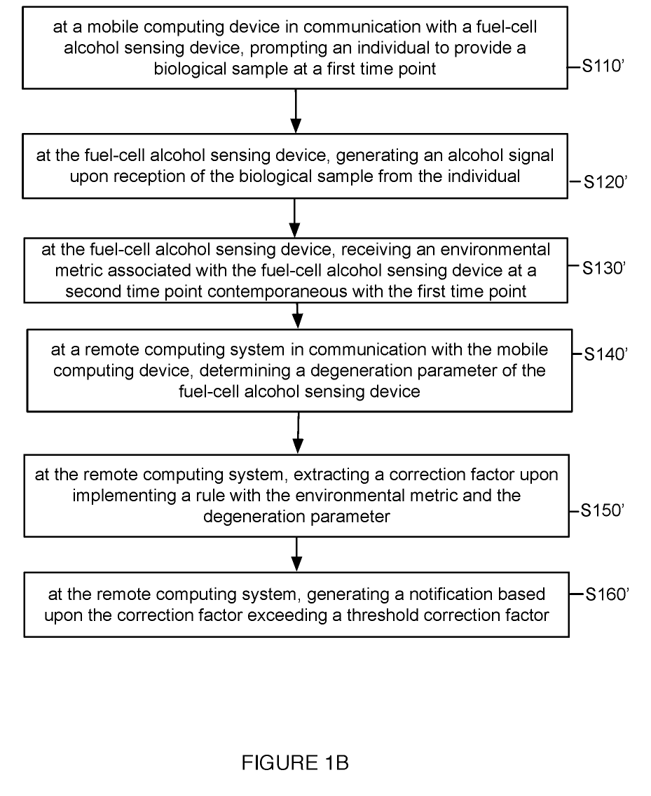

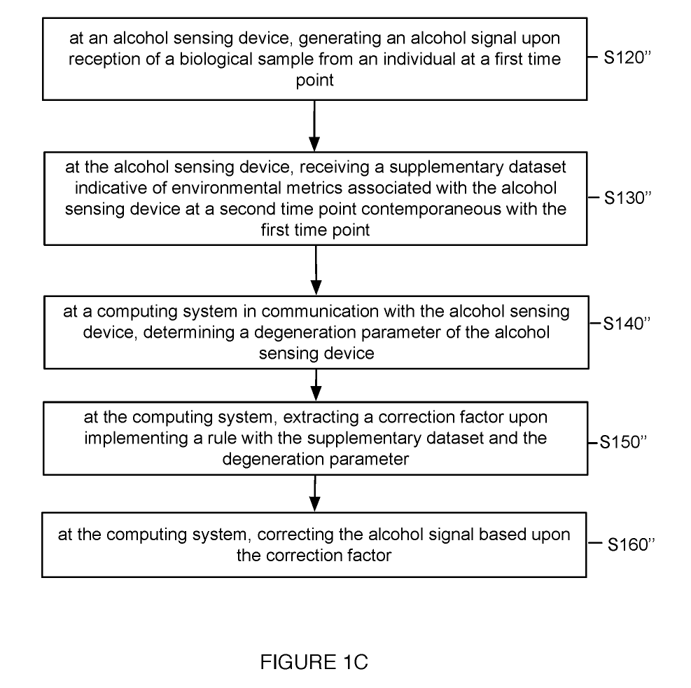

As shown in FIG. 1A, an embodiment of a method 100 for characterization of a state of an alcohol sensing device includes: receiving a biological sample S100; generating an alcohol signal upon reception of the biological sample S120; receiving a supplementary dataset S130; determining a degeneration parameter S140; extracting a correction factor S150; and performing an action based on the correction factor S160.

The method 100 functions to generate and provide an accurate measurement of a user's inebriation state based upon monitoring of sensor components of the alcohol sensing device(s). The method 100 can additionally function to notify the user of the state of their alcohol sensing device (e.g., to alert the user that a sensor of the alcohol sensing device is defective and/or requires replacement). The method 100 can additionally function to initiate the process of replacing a sensor of the alcohol sensing device, dynamically adjust subsystems of the alcohol sensing device to improve device behavior, dynamically adjust environmental conditions around the device and/or user in order to improve device behavior, and guide the user to adjust user behavior to improve device performance. The method 100 can additionally function to improve alcohol sensing device operation and/or information provided to a user of an alcohol sensing device, by accounting for factors that affect an alcohol sensing device sensor state. Preferably, the method 100 accounts for factors including one or more of: temperature, humidity/drying, sensor age, sensor wear, and other environmental factors to provide data that can be used to 1) correct analyses of alcohol sensor data, 2) manipulate alcohol sensing device operation and/or 3) inform decisions regarding sensor/device replacement at the appropriate time.

The method 100 is preferably implemented based upon aggregation and processing of sensor environment data, sensor state data, sensor output data (i.e., in relation to responses to interacting with a biological sample), sensor use data, and any other suitable data from a population of users interacting with alcohol sensing device models incorporating units of an alcohol sensing device sensor in different environments. Based upon the aggregation of data and processing of data, the method 100 can include building a database of sensor characterizations and profiles associated with environmental and/or sensor states, wherein the database is refined over time, and used to provide more accurate information in relation to alcohol sensing device test results. As such, each subsequent alcohol sensing device test can be used to refine the database and raw sensor responses can be correlated with elements of the database in order to provide more accurate information to users.

In relation to the database, aggregated data can be used as a training dataset for building models of alcohol sensing device sensor state in relation to a wide range of factors. Building models of alcohol sensing device sensor state based on training data can be performed using any other suitable machine learning algorithm(s). In variations, the machine learning algorithm(s) can be characterized by a learning style including any one or more of: supervised learning (e.g., using logistic regression, using back propagation neural networks), unsupervised learning (e.g., using an Apriori algorithm, using K-means clustering), semi-supervised learning, reinforcement learning (e.g., using a Q-learning algorithm, using temporal difference learning), and any other suitable learning style. Furthermore, the machine learning algorithm can implement any one or more of: a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naive Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolution network method, a stacked auto-encoder method, etc.), a dimensionality reduction method (e.g., principal component analysis, partial lest squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and any suitable form of machine learning algorithm. As such, models of alcohol sensing device sensor state can be used to inform analyses associated with subsequent sensor readings.

The method 100 can be implemented, at least in part, using embodiments, variations, and/or examples of the alcohol sensing device(s) described in Section 2 below, wherein the alcohol sensing devices are configured to be used outside of a “law enforcement setting”, and instead, used by users in their normal daily lives. Furthermore, variations of the method 100 can be implemented at least in part by one or more embodiments, variations, and examples of system elements described in U.S. application Ser. No. 14/169,029 entitled “Method and System for Monitoring Intoxication” and filed on 30-Jan.-2014, U.S. application Ser. No. 14/602,909 entitled “Method and System for Remotely Monitoring Intoxication” and filed on 22-Jan.-2015, U.S. application Ser. No. 14/631,125 entitled “Method and System for Monitoring Intoxication” and filed on 25-Feb.-2015, and U.S. application Ser. No. 15/375,801 entitled “Wearable System and Method for Monitoring Intoxication” and filed on 12-Dec.-2016, each of which is incorporated herein in its entirety by this reference. Variations of the method 100 can additionally or alternatively be implemented at least in part by a mobile communication device (e.g., a mobile phone, a vehicle computing system), a remote computing system (e.g., a cloud computing system, a remote server), or any other suitable systems. Furthermore, the method 100 is preferably configured for processing of data associated with fuel cell sensors; however, the method 100 can additionally or alternatively be adapted for processing of data associated with semiconductor sensors and/or any other suitable sensors for processing samples associated with intoxication or substance use.

As shown in FIG. 1B, a first specific implementation of the method 100 includes: at a mobile computing device in communication with a fuel-cell alcohol sensing device, prompting an individual to provide a biological sample at a first time point S100′; at the fuel-cell alcohol sensing device, generating an alcohol signal upon reception of the biological sample from the individual S120′; at the fuel-cell alcohol sensing device, receiving an environmental metric associated with the fuel-cell alcohol sensing device at a second time point contemporaneous (e.g., simultaneously, substantially simultaneously, in real time, in near-real time, within 1 second, within 10 seconds, within 1 minute, etc.) with the first time point S130′; at a remote computing system in communication with the mobile computing device, determining a degeneration parameter of the fuel-cell alcohol sensing device S140′; at the remote computing system, extracting a correction factor upon implementing a rule with the environmental metric and the degeneration parameter S150′; and at the remote computing system, generating a notification based upon the correction factor exceeding a threshold correction factor S160′.

As shown in FIG. 1C, a second specific implementation of the method 100 includes: at an alcohol sensing device, generating an alcohol signal upon reception of a biological sample from an individual at a first time point S120″; at the alcohol sensing device, receiving a supplementary dataset indicative of environmental metrics associated with the alcohol sensing device at a second time point contemporaneous with the first time point S130″; at a computing system in communication with the alcohol sensing device, determining a degeneration parameter of the alcohol sensing device S140″; at the computing system, extracting a correction factor upon implementing a rule with the supplementary dataset and the degeneration parameter S150″; and at the computing system, correcting the alcohol signal based upon the correction factor S160″.

### 1.1 Method—Benefits

Variations of the method 100 can confer several benefits. Variants of the method 100 can effectively alert the user that a sensor of the alcohol sensing device is in need of replacement (e.g., immediately, soon, within one week, within ten uses, etc.) upon retrieval and analysis of sensor data (e.g., an alcohol signal response curve, the shape of such a curve, environmental metrics, supplementary data, etc.) from the alcohol sensing device of the user and/or historical data from other alcohol sensing device units.

Variations of the method 100 can collect (e.g., receive and store) alcohol sensing device data over time from a population of users (e.g., in a database), along with user data and environmental data, to improve alcohol content determination (e.g., algorithms for alcohol content determination based on alcohol signals).

Variations of the method 100 can account for residual alcohol in a biological sample that is not indicative of blood alcohol content (e.g., residual alcohol in a user's mouth upon provision of a breath sample) based on supplementary data received in conjunction with the biological sample (e.g., information indicative of a time of last alcoholic beverage ingestion), and/or by other suitable techniques (e.g., receiving a non-biological air sample and measuring a humidity level, a residual alcohol level, a condensation level, etc.).

Variations of the method 100 can dynamically adjust a gain (e.g., amplification) of the generated alcohol signal and/or portions of the generated alcohol signal (e.g., regions of the response curve) in order to improve accuracy and/or precision in BAC level determination, degeneration parameter determination, and/or correction factor extraction. Variants of the method 100 can additionally or alternatively dynamically adjust hardware settings (e.g., firmware, mechanical settings, etc.) of the alcohol sensing device in response to a determined degeneration parameter (e.g., exceeding a degeneration parameter threshold) and/or an extracted correction factor.

Variations of the method 100 can automatically direct a biological sample, upon reception, from a primary (e.g., default) sensor to a secondary (e.g., redundant, additional) sensor in cases wherein the primary sensor has degraded beyond a degradation threshold.

Variants of the method 100 can improve the technical field of blood alcohol content determination by automatically adjusting for sensor degeneration, collecting real-time (e.g., near real-time) environmental data including factors (e.g., parameters) that can affect the sensor reading and correcting therefor (e.g., in conjunction with a cloud-hosted computation and/or data model).

Related variants of the method 100 can improve the technical field of remote blood alcohol content determination by preventing false negatives and/or false positives being reported to a monitoring entity due to erroneous measurements resulting from contributing environmental factors (e.g., humidity, temperature, etc.).

### 1.2 Method—Sample Reception

Block S110 recites: receiving a biological sample, which functions to obtain a sample of biological material indicative of a BAC level of a user for use in subsequent blocks of the method 100. Block S110 is preferably performed at the alcohol sensing device, and can be performed in cooperation with a mobile computing device or other portions of a computing system. Alternatively, Block S110 can be performed or implemented at any suitable location. Block S110 can include receiving the biological sample from the user, an individual, or any other suitable person; alternatively, the biological sample can be received from a laboratory, a fluid handling robot, or any other suitable entity. The biological sample can include a biological sample, a transdermal fluid sample, a saliva sample, or any other suitable biological material from a user that can contain alcohol and/or derived compounds originating from alcohol in proportion to a BAC level of the user.

In one example of Block S110, an individual deposits a biological sample into a cavity of a breathalyzer by way of an aperture in the cavity. The biological sample is directed to a fuel-cell sensor of the device (e.g., by a bellows, by internal contours of the breathalyzer cavity) and is thereby received at the breathalyzer. In another example of Block S110, transdermal fluid is excreted into a volume of a skin-adjacent cavity of a wrist-worn alcohol sensor, and is received at a fuel-cell sensor of the wrist-worn alcohol sensor. In other example implementations of Block S110, the biological sample can be received in a manner and/or form as described in U.S. application Ser. No. 15/294,998, entitled “Method and System for Monitoring Intoxication” and filed 17-Oct.-2016,U.S. application Ser. No. 14/973,227, entitled “Method and System for Remotely Monitoring Intoxication” and filed 17-Dec.-2015, U.S. application Ser. No. 15/294,317, entitled “Method and System for Monitoring Intoxication” and filed 14-Oct.-2016, U.S. application Ser. No. 15/205,876, entitled “Method and System for Drunk Driving Prevention” and filed 08-Jul.-2016, and U.S. application Ser. No. 15/375,801, entitled “Wearable System and Method for Monitoring Intoxication” and filed 12-Dec.-2016, each of which is incorporated in its entirety herein by this reference. However, Block S110 can be otherwise performed.

In more detail, related to an example of Block S110 incorporating a breathalyzer, receiving the biological sample can include receiving the biological sample at a cavity of an alcohol sensing device, wherein the cavity includes a first aperture and a second aperture configured to facilitate breath intake and outflow, respectively. In the specific example, the cavity is in communication with a fuel cell sensor that receives a metered volume of the biological sample, wherein the fuel cell sensor facilitates generation of an alcohol signal by an electrochemical process. As such, in the specific example, the alcohol signal comprises an electrical parameter (e.g., voltage) having a profile (e.g., response curve) related to time, that can be detected and processed to determine a value of an intoxication metric indicative of the sobriety of the individual. Variations of the specific example can, however, include generation of any other suitable type of electrical signal in response to a received biological sample from the individual.

In alternative variations of Block S110 of the method 100, receiving a biological sample from the individual can be substituted with or supplemented with receiving any one or more of: urine samples, blood samples, interstitial fluid samples, and any other suitable sample (e.g., from a transdermal fluid sensor) that can be used to assess the user's substance use. For example, receiving the biological sample can include receiving the biological sample from the individual at a skin contact region of the fuel-cell alcohol sensing device (e.g., wherein the biological sample is a transdermally excreted fluid sample). Furthermore, in relation to any of the above types of samples, samples used to determine sobriety and substance usage by the individual are preferably received from the user in a non-invasive manner; however, samples can additionally or alternatively be received in a minimally invasive or invasive manner. Furthermore, in some variations, a signal generated in response to a received sample can be generated without directly collecting a sample from the individual. For example, a signal can be generated in an indirect manner, as derived from an interaction between a stimulus and the user's body (e.g., spectrometer-based analysis of light transmitted from a user's blood vessels).

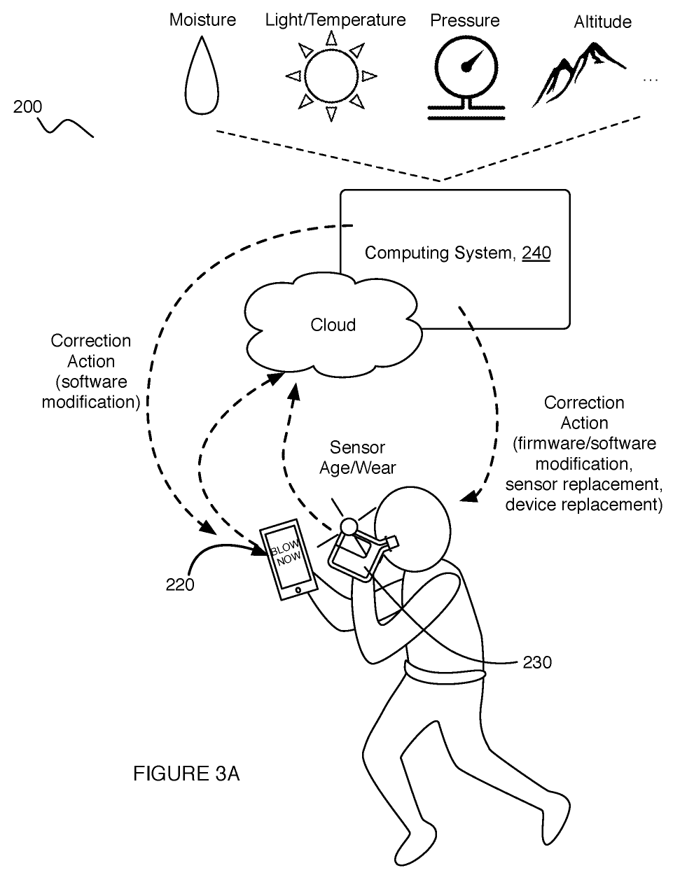

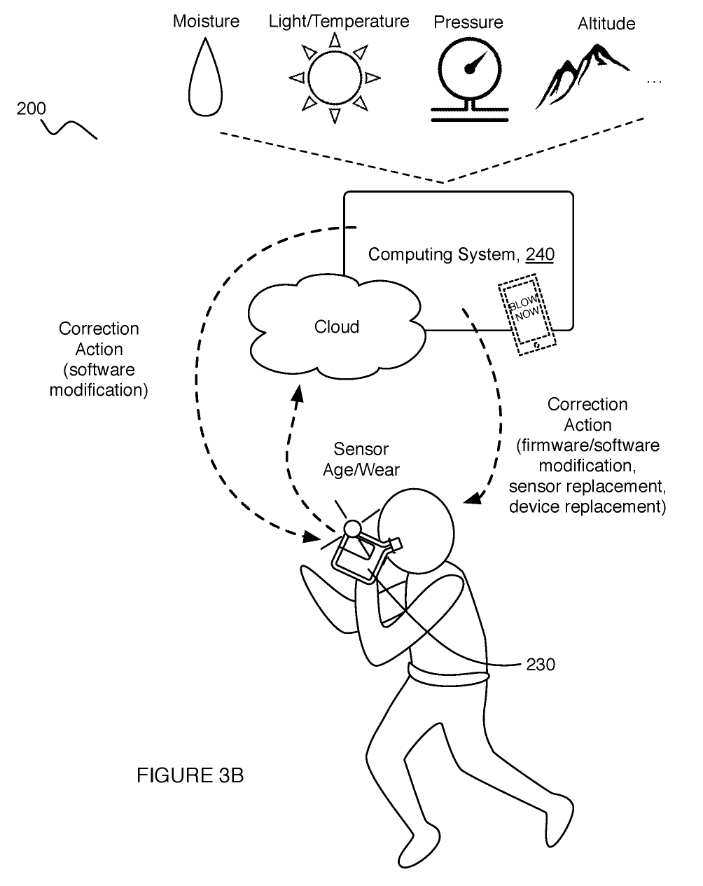

Block S110 can optionally include Block S112, which recites: by way of an application executing at a mobile computing device in communication with an alcohol sensing device, prompting an individual to provide a biological sample at a time point, which functions to prompt the individual to provide the biological sample in association with reception of supplementary data (e.g., a supplementary dataset, environmental data, sensor age data, etc.) in Block S130. Block S112 preferably includes using an application executing at a mobile computing device in communication with the alcohol sensing device and a computing system, wherein the application guides the user in providing the biological sample. However, Block S112 can additionally or alternatively be implemented using any other suitable computing device (or non-computing entity) that can be used to prompt the user to provide a biological sample. In Block S112, prompting is preferably performed using one or more of: display functions of a display of the mobile computing device (or alcohol sensing device), an example of which is shown in FIG. 3A, speaker functions of an audio-output element of the mobile computing device (or alcohol sensing device), haptic functions of an actuation element (e.g., vibration motor) of the mobile computing device, visual signal functions of a light emitting element (e.g., LED) of the mobile computing device (or alcohol sensing device), and/or any other suitable function of the mobile computing device (or alcohol sensing device).

In one example of Block S112, a native application executing at the mobile computing device can provide a graphic and/or textual message, using the display of the mobile computing device, to cue the user to provide a biological sample upon pairing of the mobile computing device with an associated alcohol sensing device. In a specific example, the native application can implement the display of the mobile computing device in order to display information pertaining to the alcohol sensing device that has been paired with the mobile computing device, along with textual messages that indicate an initiation time point of biological sample provision and/or an appropriate duration of biological sample provision, in order to achieve a suitable biological sample. Additionally or alternatively, prompting in Block S112 can be performed using the alcohol sensing device described in subsequent portions of the disclosure, for instance, using LED signals to prompt sample provision. Additionally or alternatively, prompting in Block S112 can implement a haptics system (e.g., including a vibrating element) that functions to prompt the individual to provide a biological sample. Additionally or alternatively, prompting in Block S110 can be performed in a non-electronic format, for instance, by way of an interaction (e.g., a verbal interaction, an interaction in writing, etc.) between the user and another human entity associated with the user.

In Block S112, prompting the individual to provide the biological sample can have associated time and/or location based condition associated with states in which it would be advantageous to acquire additional data. For instance, in relation to acquiring sensor data in a specific environment, Block S112 can include Block S114, which recites: based upon identification of an environment or a sensor state of the alcohol sensing device associated with the individual, prompting the individual to provide the biological sample. In variations, the environment can be an environment in which a low amount of alcohol sensing device sensor data has been acquired, such that Block S114 prompts individuals to provide biological samples whenever they are in environments in which there is scarcity of data. Alternatively, Block S114 can include prompting individuals to provide biological samples whenever they are in any other suitable environment. In variations, the sensor state can be a sensor state at which a low amount of alcohol sensing device sensor data has been acquired, such that Block S112 prompts individuals to provide biological samples whenever their alcohol sensing device sensors are at states in which there is scarcity of data (e.g., high states of wear, high ages, etc.).

Additionally or alternatively, Block S112 can be implemented using display (or other output elements) of the alcohol sensing device.

Satisfaction of the condition(s) for prompting the individual in Block S112 can be detected using one or more sensor or electronics modules of devices of the computing system or devices of the individual in communication with the computing system. In one example, satisfaction of an environmental condition can be determined using a temperature sensor of the alcohol sensing device and/or mobile computing device. Satisfaction of the environmental condition can additionally or alternatively include using a GPS module of the alcohol sensing device or mobile computing device to identify a location of the alcohol sensing device sensor, and then retrieving local environmental conditions (e.g., weather, temperature, humidity, etc.) associated with the location. Satisfaction of a sensor state condition can be assessed by using identification information of the alcohol sensing device unit, and retrieving an age of the sensor (e.g., including batch number, etc.). Satisfaction of a sensor state condition can additionally or alternatively be assessed by using identification information of the alcohol sensing device unit, and retrieving a number of uses of the alcohol sensing device (e.g., in a manner that indicates wear of the alcohol sensing device sensor). Satisfaction of the condition(s) can, however, be determined in any other suitable manner.

Furthermore, prompting in Block S112 can additionally or alternatively be performed for calibration purposes. For instance, in one variation, an individual can be prompted to provide a zero-alcohol content sample (e.g., a 0.00% BAC sample), wherein subsequent data from alcohol signal analysis is analyzed (e.g., in near real time) to diagnose sensor performance, remaining life of the sensor, sensor drift, and/or any other suitable parameter(s) in line with subsequent blocks of the method 100 (e.g., Block S160) described in more detail below. However, prompting can be performed in any other suitable manner, under any other suitable condition(s).

### 1.3 Method—Signal Generation

Block S120 recites: generating an alcohol signal upon reception of the biological sample at a time point, which functions to generate a signal that can be characterized in subsequent blocks of the method 100, in order to determine the alcohol content of the biological sample. Block S120 is preferably performed at the alcohol sensing device based on the received biological sample, but can alternatively be based on any suitable sample. Block S120 can additionally include generating a series of alcohol signals upon reception of a series of biological samples at a series of time points (e.g., at a wearable alcohol sensing device in extended contact with the skin of a user, acquiring transdermally excreted fluid samples at regular intervals).

Block S120 can additionally or alternatively include Block S125, which recites: transmitting an alcohol signal, derived from reception of the biological sample from the individual, to at least one of the computing system (e.g., remote server) and the mobile computing device implemented in embodiments of the method 100. Block S120 functions to receive a sample from the user from which a signal can be generated and a profile of the signal can be characterized, in subsequent blocks of the method 100.

In Block S120, the alcohol signal is preferably generated at a fuel cell sensor that enables measurement of the individual's blood alcohol content (BAC), and/or other intoxication factor indicative of sobriety, by an electrochemical process. In relation to the fuel cell sensor, generating the alcohol signal can include producing an electrical current in response to oxidation of alcohol carried in the biological sample provided by the user, wherein the magnitude of the produced electrical current varies in a predictable manner according to the amount (e.g., relative volume) of alcohol carried in the biological sample. In more detail, the signal can be amplified and then digitized to produce a digital signal, wherein the digital signal is processed (e.g., in relation to gain, sample rate, windowing, etc.) by the mobile computing device and/or in other computing systems (e.g., a cloud-based computing system), and/or stored. In an alternative variation, generating the alcohol signal can be implemented at a semiconductor sensor that produces a change in electrical resistance in response to an alcohol-dioxide reaction, wherein the magnitude of the change in resistance varies in a predictable manner according to the amount (e.g., relative volume) of alcohol carried in the biological sample. In more detail, the signal can be amplified and then digitized to produce a digital signal, wherein the digital signal is processed (e.g., in relation to gain, sample rate, windowing, etc.) by the mobile computing device and/or in other computing systems (e.g., a cloud-based computing system), and/or stored. In other variations however, Block S120 can additionally or alternatively include generating a alcohol signal at a spectrophotometer configured to produce a signal in response to absorbed or emitted light from alcohol molecules carried in the biological sample from the user. Generating the alcohol signal in Block S130 can, however, include generating a signal at any suitable element configured to respond to alcohol in a sample from the user, wherein the signal(s) generated is(are) processed and/or digitized (e.g., in relation to gain, sampling rate, windowing, etc.) for analysis purposes.

In variations, Block S120 can include generating an alcohol signal based on a transfer function between the signal generated at a fuel-cell sensor and the BAC metric of a user. For example, in the context of a transdermal alcohol sensing device, Block S120 can include generating an alcohol signal indicative of the BAC of a user based on a signal derived from transdermal alcohol content. This can include implementing a rule to transform a transdermal alcohol content signal into an alcohol signal indicative of blood alcohol content (e.g., evaluating a known function, determining a function according to a model and evaluating the function, querying a database containing a set of numerical relationships between transdermal alcohol content values and blood alcohol content values, etc.).

While some embodiments, variations, and examples of Blocks S110-S120 are described above with respect to incorporation of a mobile computing device, alternative variations of Blocks S110-S130 can omit involvement of or otherwise reduce reliance upon a mobile computing device in initiating biological sample provision and/or reception.

### 1.4 Method—Supplementary Data Reception

Block S130 recites: receiving a supplementary dataset, which functions to allow sensor response data to be associated with supplementary data that can impact sensor response. The supplementary dataset is preferably indicative of environmental conditions (e.g., environmental metrics) associated with the alcohol sensing device sensor proximal in time to the time point of biological sample provision, which can function to allow sensor response data to be associated with at least one environmental factor that affects sensor response. Block S130 can include, in variations, receiving an environmental metric (e.g., directly). For example, Block S130 can include detecting a humidity level at a humidity sensor of the fuel-cell alcohol sensing device. Preferably, Block S130 includes reception of one or more of: environmental temperature data, environmental humidity data, environmental barometric pressure data, environmental altitude data, environmental light data, and any other suitable data associated with an environmental parameter that could affect sensor performance. In variations, receiving environmental data can comprise receiving environmental data (e.g., metrics) directly from one or more of: sensors of the alcohol sensing device, sensors of the mobile computing device, and/or any other sensors in the environment of the individual providing a biological sample. In alternative variations, environmental data can be generated in an indirect manner, upon retrieving a location of the alcohol sensing device (e.g., using a GPS module, using a triangulation system, etc.) and extracting environmental data based upon the location of the alcohol sensing device.

In a specific example, Block S130 can include receiving environmental temperature data from a temperature sensor (e.g., thermocouple, etc.) integrated with one or more of the alcohol sensing device and mobile computing device. In an alternative specific example, Block S130 can include extracting a location of the alcohol sensing device from a GPS module (e.g., a GPS module of a mobile computing device in communication with the alcohol sensing device, a GPS module of the alcohol sensing device), and then extracting local temperature data based upon the retrieved location (e.g., from a weather information provider). Additionally or alternatively, in another specific example, Block S130 can include receiving environmental humidity data (e.g., in terms of dew point, in terms of relative humidity, in terms of parts per million, etc.) from a humidity sensor (e.g., moisture sensor, capacitance sensor, hygrometer, etc.) integrated with one or more of the alcohol sensing device, mobile computing device, and computing system. In an alternative specific example, Block S130 can include extracting a location of the alcohol sensing device from a GPS module (e.g., a GPS module of a mobile computing device in communication with the alcohol sensing device, a GPS module of the alcohol sensing device), and then extracting local humidity data based upon the retrieved location (e.g., from a weather information provider). Additionally or alternatively, in another specific example, Block S130 can include receiving environmental pressure data from a pressure sensor integrated with one or more of the alcohol sensing device, mobile computing device, and computing system. In an alternative specific example, Block S130 can include receiving a supplementary dataset that includes an operational lifetime of a sensor of the alcohol sensing device. In an alternative specific example, Block S130 can include measuring a temperature at a temperature sensor of the fuel-cell alcohol sensing device, and measuring a humidity level at a humidity sensor of the fuel-cell alcohol sensing device, wherein receiving the supplementary dataset includes receiving the temperature and humidity level. In an alternative specific example, Block S130 can include extracting a location of the alcohol sensing device from a GPS module (e.g., a GPS module of a mobile computing device in communication with the alcohol sensing device, a GPS module of the alcohol sensing device), and then extracting local pressure data based upon the retrieved location (e.g., from a weather information provider). Additionally or alternatively, in another specific example, Block S130 can include extracting environmental altitude data (e.g., from an altimeter of one or more of the mobile computing device and the alcohol sensing device), Additionally or alternatively, in another specific example, Block S130 can include extracting environmental light data (e.g., from camera units of one or more of the mobile computing device and the alcohol sensing device). However, Block S130 can include retrieving or receiving any other suitable environmental data, by any other suitable means, in relation to the supplementary dataset of Block S130.

In variations, Block S130 can include generating command instructions based on the supplementary dataset, and can further include transmitting the command instructions from the computing system to an environmental control system that adjusts the environmental metrics (e.g., as received in the supplementary dataset) associated with the alcohol sensing device in response to the command instructions. In a specific example implementation, Block S130 can include sending commands to a connected thermostat device (e.g., a Nest device) to reduce or increase the temperature set point of the thermostat based on received temperature data, in order to improve the operation of the alcohol sensor.

### 1.5 Method—Degeneration Determination

Block S140 recites: determining a degeneration parameter of a sensor of the alcohol sensing device, which functions to assess the state of the alcohol sensing device and the present and/or future capability of the sensor to provide accurate measurement of the alcohol content of received biological samples. Determining the degeneration parameter is preferably performed by a remote computing system, but can additionally be performed in whole or in part by the alcohol sensing device, the mobile computing device, or any other suitable computing device. Determining the degeneration parameter is preferably based on characteristics of the generated alcohol signal (e.g., the amplitude of the signal, shape of the signal response curve versus time, etc.), but can alternatively be based on any suitable characteristics of the sensor. The degeneration parameter is preferably an indicator of the physical state of a sensor (or sensors) of the alcohol sensing device (e.g., an absolute age of the sensor, a relative age of the sensor, sensor efficiency, hydration level of the sensor or a membrane of the sensor, etc.), but can additionally or alternatively be any suitable parameter (e.g., a number of uses of the sensor) indicative of sensor state. Determining the degeneration parameter can, in variations, include measuring a shape of the alcohol signal (e.g., of the response curve) and correlating the shape with the degeneration parameter (e.g., via a lookup table or database stored in the cloud or at the mobile device, via an analysis of shape features such as maximum or minimum magnitude, etc.). In one example, the received alcohol signal is a zero alcohol content response curve (e.g., nominally corresponding to a 0.00% BAC), and correlating the shape of the zero alcohol content response curve with the degeneration parameter includes comparing the zero alcohol content response curve to an ideal zero alcohol content response curve (e.g., a reference 0.00% BAC curve stored at a cloud computing system and transmitted to the mobile device for comparison and correlation at the mobile device).

Block S140 can optionally include Block S142, which recites: receiving identifying information, including an age of the alcohol sensing device, which functions to allow sensor response data to be associated with at least one sensor state factor that affects sensor response (e.g., in relation to aging or wearing of the sensor(s)). Preferably, Block S142 includes reception of identifying information that can be used to extract data related to one or more of: number of uses of the alcohol sensing device/alcohol sensing device sensor, on-off cycling of the alcohol sensing device/alcohol sensing device sensor, idling states of the alcohol sensing device/alcohol sensing device sensor, charging states of the alcohol sensing device, energy use associated with the alcohol sensing device sensor, battery state of the alcohol sensing device during use of the alcohol sensing device sensor, overall age of the sensor (in relation to a reference point), amount of mechanical perturbation of the sensor (e.g., vibration amount as assessed from an accelerometer), thermal perturbation of the sensor (e.g., in relation to thermal cycles), and any other suitable data. Such data can be retrieved using an identifier of the alcohol sensing device (e.g., serial number), and/or data aggregation units of one or more of the alcohol sensing device and mobile computing device, wherein the data aggregation units are configured to collect, store, and/or transmit device state information.

Data received in one or more of Blocks S130 and S140 can additionally or alternatively include any other suitable data related to sensor response and/or modified sensor performance. For instance, Blocks S130 and/or S140 can include reception of data related to oxidation of sensors, foreign substances (e.g., mouth wash, fresheners, food, liquids, paint fumes, varnish, etc.) present in the environment of the sensor or user, user behaviors that could affect responses (e.g., cigarette smoking, dental accessory use that results in residual mouth alcohol, low carbohydrate dieting, etc.), user conditions that could affect responses (e.g., gastroesophageal reflux disease that causes undigested alcohol to affect readings, burping, prescription medication use, etc.), and/or any other suitable data.

In relation to data transmission in Block S130 and/or S140, transmission can include streaming data over a suitable wired or wireless link (e.g., BlueTooth, BlueTooth LE, Wi-Fi, Cellular, etc.) to the computing system (e.g., with at least some portions of the computing system implemented in a cloud-based computing subsystem and/or mobile device). As such, data can be transmitted in near-real time (e.g., when associated networks become available), such that data is received, stored, and/or processed as soon as practically possible for each alcohol sensing device test operation. In relation to data transmission and storage, data can be accessed (e.g., through a web page or application) for analysis. Additionally or alternatively, data from specific users can be anonymized or used only with user permission. Additionally or alternatively, data can be tagged such that data can be queried. Furthermore, data of Block S130 and/or S140 can be collected at any suitable frequency in relation to biological sample provision in Blocks S110 and S120. For instance, in a specific example, supplementary and/or other data can be collected at regular or irregular intervals (e.g., on the order of seconds, on the order of minutes, on the order of hours, on the order of days, etc.). However, data can be stored and transmitted in any other suitable manner.

### 1.6 Method—Correction Factor Extraction

Block S150 recites: extracting a correction factor upon implementing a rule with the supplementary dataset and the degeneration parameter, which functions to calculate how the alcohol signal can be adjusted according to information in the supplementary dataset and the degeneration parameter. Extracting the correction factor can include computing the correction factor, calculating the correction factor, selecting (e.g., looking up) the correction factor, evaluating the correction factor, or any other suitable process for deriving the correction factor. The correction factor is preferably a multiplicative factor that can subsequently be used to scale (e.g., multiply) the alcohol signal to account for exogenous factors (e.g., factors described by the supplementary dataset and/or the degeneration parameter). Additionally or alternatively, the correction factor can be a single valued function that can be evaluated at each value in a set of values of the alcohol signal (e.g., wherein the signal is a vector of points ‘x’, the correction factor is a function ‘f’, and can be evaluated to produce a corrected signal ‘f[x]’), a transfer function that can be convolved with the alcohol signal (e.g., wherein the signal is a transformed vector of points ‘X’, the correction factor is a transfer function ‘F’, and the corrected signal is the convolution of ‘F’ and ‘X’) , or any other suitable mechanism of correcting the alcohol signal. Implementing a rule can include: looking up a value in a table (e.g., stored in a database), evaluating a function (e.g., a single-valued single-variable function, a single-valued multi-variable function), transforming signals into a frequency domain and performing frequency domain operations followed by applying a reverse transform, comparing a shape of an alcohol signal response curve and shape of a reference response curve, or any other suitable rule or rules implementation. In a specific example, Block S150 includes evaluating an extraction function, wherein an output of the extraction function includes the correction factor and wherein inputs of the extraction function include the degeneration parameter and features of the supplementary dataset (e.g., temperature values, sensor age, etc.).

A specific example implementation of Block S150 includes extracting a correction factor from a comparison of a shape of an alcohol signal response curve with a reference response curve. The shape can include various shape aspects, such as peaks, troughs, decay rate, area under the curve, shapes of different curve regions (e.g., beginning, middle, end, arbitrary regions, etc.), and any other suitable aspects. In some example implementations, Block S150 can include measuring a relative flatness of the alcohol signal response curve compared to a reference response curve (e.g., the correction factor includes a ratio of the slope of a portion of the alcohol signal response curve to the slope of a portion of the reference response curve).

In another specific example of Block S150, the received environmental metric is within a first range of values, the determined degradation parameter is within a second range of values, and the first and second range of values defines a two-dimensional parameter space (e.g., a function wherein the inputs are an environmental metric value and a degeneration parameter value and the output is a correction factor). in this example, extracting the correction factor upon implementing the rule includes selecting the correction factor from the two-dimensional parameter space based on the received environmental metric (e.g., the value of the environmental metric) and the degeneration parameter (e.g., the value of the degeneration parameter).

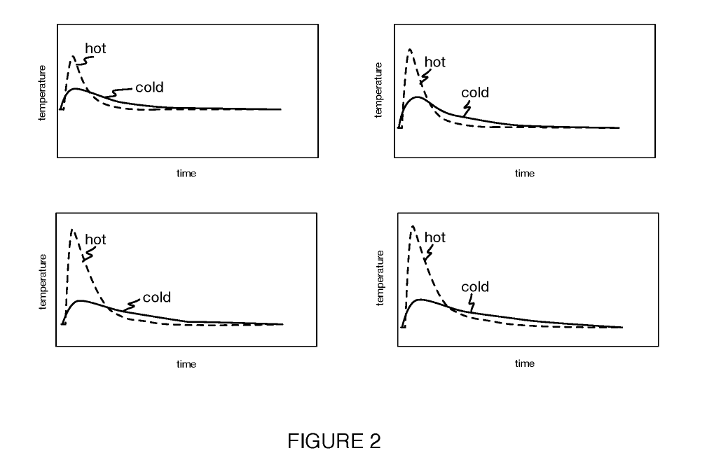

Block S150 can optionally include Block S152, which recites: based upon the supplementary dataset and the identifying information, correlating a profile of the alcohol signal to a correction factor of the alcohol sensing device sensor. The profile is preferably a shape of the alcohol signal, but can additionally or alternatively be any suitable characteristics of the alcohol signal. In relation to the trained models of sensor state described above, parameters associated with the alcohol signal can be extracted and used as inputs for the trained models, in order to generate outputs related to the adjusted sensor state and/or suitable correction operations indicated in Block S160 below. In relation to the correlation of Block S152, extracted parameters of sensor response signal profiles (examples of which are shown in FIG. 2) can include one or more of; environmental factors (e.g., temperature, pressure, humidity, light, etc.) from the supplementary dataset; sensor state factors (e.g., number of uses, on-off cycling history, idling state history, charging state history, energy use, battery state history, overall age of the sensor, amount of mechanical perturbation of the sensor, thermal perturbation history of the sensor, etc.); sensor curve parameters (e.g., peak amplitude, equilibrium amplitude, rising slopes, falling slopes, position of feet of peaks, inflection points, areas under the curve, etc.) directly extracted from the sensor signal response profile; other parameters indicated above (e.g., user behavior, user conditions, presence of foreign substances, etc.); and or any other suitable parameters that can be used as inputs into trained models. As shown by way of example in FIG. 2, the response curve (e.g., a shape of the response curve) can vary with temperature (e.g., at lower temperatures, response curves can have a lower peak and longer tail); humidity of the local environment of the sensor and/or dehydration of the sensor can have similar or compounded effects on the response curve shape. Curve shapes can additionally or alternatively vary in a nonlinear manner as a function of multiple environmental metrics and alcohol content represented by the signal. In a specific example of Block S150, the shape of the response curve is compared alongside environmental metrics received in the supplementary dataset to determine that the sensor is dehydrated (e.g., wherein the correction factor includes information that the sensor is dehydrated). In an alternative specific example, the shape of the response curve is compared alongside environmental metrics received in the supplementary dataset to determine the age of the sensor (e.g., wherein the correction factor includes an age of the sensor).

In relation to near-real time streaming of sensor response profile data, characteristic parameters of the profile (e.g., curve aspects, shapes) can be processed in real time or near-real time. As such, processing can be used to provide projected or predicted profile aspects (e.g., aspects related to the peak signal, aspects related to the baseline signal, an indication of progress from peak to baseline signal acquisition, etc.), predicted/projected intoxication metric values (e.g., BAC values), and/or any other suitable information. In another specific example, near-real time streaming can be used to analyze initial portions of a sensor signal profile to determine user behaviors (e.g., not waiting a certain period of time after drinking to provide a biological sample) associated with poor accuracy, in order to provide notifications in association with variations of Block S160 of the method 100 described below.

Any identified correction factors that are produced as outputs of such trained models can thus be used to select appropriate correction operations (e.g., to correct the alcohol signal) as described in more detail in Block S160 below.

### 1.7 Method-Action Performance

Block S160 recites: performing an action based on the correction factor, which functions to generate a response to the extracted correction factor. Block S160 can additionally function to notify the user of information associated with the correction factor, and/or to automatically correct the alcohol signal based on the correction factor. Block S160 can include: generating a notification based on the correction factor S162; and correcting the alcohol signal based on the correction factor S165.

Block S162 recites: generating a notification based on the correction factor, which functions to notify the user of pertinent information related to the extracted correction factor. Variants of generating a notification can include displaying, delivering, and/or rendering the notification (e.g., at the mobile computing device, at the alcohol sensing device, etc.). In one example, the notification can include an indication that the alcohol sensing device requires a replacement sensor (e.g., a push notification at an application executing at the mobile device that the alcohol sensing device requires a replacement sensor).

Additionally or alternatively, Block S162 can include generating an alert configured to inform the individual or other entity regarding a state of interest of the alcohol sensing device or sensor. In a specific example, Block S162 can comprise generating an alert that indicates that the individual needs a new alcohol sensing device/sensor. In another specific example, Block S162 can comprise generating an alert that the alcohol sensing device of the individual is located in an environment (e.g., a hot car) that is bad for sensor performance or maintenance. Additionally or alternatively, Block S162 can include generating an alert that indicates that the user should provide another biological sample when the user relocates to an environment more conducive to accurate biological sample testing. However, variations of Block S162 can additionally or alternatively include generating any other suitable type of alert.

Block S162 can additionally include generating a notification in the context of impaired (e.g., drunk) driving. For example, Block S162 can include generating a notification that a computation of blood alcohol content may be of low accuracy, based on the extracted correction factor, and therefore not suitable for determining whether the user's blood alcohol content is within legal limits (e.g., until the sensor is replaced, until the user is in a more hospitable environment for proper sensor operation, until the user is outside of a hot vehicle, etc.).

Block S165 recites: correcting the alcohol signal based on the correction factor, which functions to perform an operation specific to the current state of the alcohol sensing device sensor, and modify the alcohol signal to account for factors such as those described above (e.g., the supplementary dataset, environmental metrics, degradation parameter, etc.) to produce a corrected alcohol signal. Correcting the alcohol signal can include, in variations, evaluating a correction function, wherein an input of the correction function includes the correction factor and an output of the function includes a corrected alcohol signal. In a specific example, evaluating the correction function can include scaling the alcohol signal by the correction factor to generate the corrected alcohol signal. In an alternative specific example, the correction factor includes a corrective response curve, and correcting the alcohol signal based upon the correction factor includes convolving the corrective response curve with the alcohol signal to generate a corrected alcohol signal.

A specific example of Block S165 can include performing a correction operation configured to improve operation of the alcohol sensing device, based upon the correction factor. Preferably, Block S165 includes performing an operation that improves or otherwise increases the accuracy of alcohol sensing device sensor test information provided to the user in a current or subsequent test using the alcohol sensing device. Alternatively Block S165 can include replacing one or more of the alcohol sensing device sensor and the alcohol sensing device unit associated with the extracted correction factor, in order to provide the individual with a unit that functions properly (e.g., provides alcohol content data within an acceptable accuracy range, within 0.1%, within 0.01%, etc.).

In one variation, the correction operation of Block S165 can comprise automatically updating or customizing the firmware of the alcohol sensing device/sensor associated with the individual in order to provide test information that accurately reflects the intoxication parameter (e.g., blood alcohol content) associated with the biological sample. In a specific example of this variation, a user's alcohol sensing device firmware can be updated automatically to compensate for environmental factors in a detected geographic location of the alcohol sensing device and/or any other suitable factor.

In another variation, the correction operation of Block S165 includes operating the alcohol sensing device between two modes. In the first mode, the alcohol sensing device directs incoming biological samples toward a primary sensor, and in the second mode, the alcohol sensing device directs incoming biological samples toward a secondary sensor. In this variation, correcting the alcohol signal based on the correction factor (e.g., performing the correction operation) includes: processing the correction factor with a criterion (e.g., comparing the correction factor to a threshold value); transitioning the alcohol sensing device between the first mode and the second mode upon detection that the correction factor fails to satisfy the criterion (e.g., the correction factor is too high and/or exceeds a threshold value, indicating that the primary sensor has worn out or degraded); and generating a second alcohol signal upon reception of a second biological sample at the second sensor.

Additionally or alternatively, in another variation, Block S165 can include automatically updating the software associated with digitized signal processing on the mobile computing device of the individual, with or without updating the firmware. Updating software modules can include updating software to perform modified signal processing operations (e.g., signal transformation operations associated with scaling, culling, windowing, amplification, etc.) in order to generate test information that accurately reflects the intoxication parameter (e.g., blood alcohol content) associated with the biological sample. As such, in a specific example, device firmware can be static, while Block S160 includes updating device software.

Additionally or alternatively, in another variation, Block S165 can comprise replacing one or more of the alcohol sensing device and the alcohol sensing device sensor. For instance, Block S160 can be used track alcohol sensing device sensor age and to recover worn alcohol sensing device sensors for studying. Alternatively, variations of Block S160 can be used to automatically provide individuals with new alcohol sensing device units/sensors according to a subscription model.

In another variation, Blocks S165 and S162 can include rendering, at a display of the computing system, a blood alcohol metric computed based upon the corrected alcohol signal. Blocks S162 and S165 can, in variations, be performed iteratively and/or dynamically; for example, the user can be notified that the environment is too hot for proper device operation, automatically detect that the user has moved to a colder environment and re-prompt the user to provide a second biological sample, and then compute a corrected alcohol signal based on the second biological sample; in this example, if the correction factor is still outside of a nominal range due to the temperature of the user's environment, the sequence can be repeated.

In a specific example of the method 100, the user is prompted to provide a “zero alcohol” biological sample (e.g., a biological sample provided when the user has not consumed alcohol within a time period long enough such that no alcohol is detectable in the sample). In this example, the zero alcohol content biological sample is received as a breath sample at a breathalyzer of the user; however, in related examples, the zero alcohol content biological sample can be received as a transdermal fluid sample at an ankle-worn alcohol sensing device of the user. The breathalyzer generates an alcohol signal and transmits the alcohol signal to a paired mobile device, which transmits the alcohol signal to a cloud computing system. At the cloud computing system, the response curve of the zero alcohol content signal is compared to a stored response curve of a reference zero alcohol content signal (e.g., the signal a sensor without any degradation would produce in response to a zero alcohol content signal) and it is determined, based on this comparison, that the sensor of the user's breathalyzer needs to be recalibrated. In related or alternative examples, the zero alcohol content signal can be completely flat (e.g., have no appreciable amplitude change versus time), which can indicate that there is a mechanical malfunction of the breathalyzer and/or a fully degraded (e.g., ready for replacement) fuel-cell sensor. The cloud computing system generates a notification that the user's breathalyzer needs to be recalibrated based on the comparison as described above, and transmits the notification to the mobile device of the user where it is displayed to the user.

In a related specific example of the method 100, the user is prompted to provide a biological sample that has a nonzero alcohol content (e.g., a biological sample provided when the user has consumed alcohol within a time period short enough such that alcohol is detectable in the sample). The alcohol sensing device generates an alcohol signal and transmits the alcohol signal in real time (e.g., streams the signal as it is generated) to a paired mobile device, which transmits the alcohol signal to a cloud computing system. The paired mobile device also generates a preliminary BAC assessment based on the received alcohol signal. At the cloud computing system, the shape of the response curve of the signal is compared to a database of stored response curve shapes corresponding to various factors and combinations of factors affecting response curve shape (e.g., temperature, humidity, sensor degradation/degeneration, etc.) and it is determined, based on this comparison, that the sensor needs to be recalibrated and that the user's BAC cannot be determined without recalibration. The cloud computing system generates a notification that the user's breathalyzer needs to be recalibrated based on the comparison as described above, and transmits the notification to the mobile device of the user where it is then transmitted to the alcohol sensing device and displayed to the user at a display of the alcohol sensing device (e.g., a message stating “calibration needed” is displayed).

Additionally or alternatively, variations of the method 100 can include switching between receiving and/or determining information that is optimized for speed and information that is optimized for accuracy. Switching is preferably based upon received user input, but can alternatively be performed automatically. User input can be received via an application (e.g., executing at the user's mobile device), via an input of the alcohol sensing device (e.g., a button), or otherwise received. For instance, information optimized for speed can be based upon analyses of a peak of an alcohol signal, while information that is optimized for accuracy can be based upon analysis of the integrated area of an alcohol signal. Alternatively, the method 100 can include automatically switching between different modes (e.g., speed mode, accuracy mode, peak analysis mode, integrated area analysis mode) based upon detected environmental metrics, received supplementary datasets, extracted correction factors and/or any other suitable factors.

Blocks of the method 100 can be repeated in order to build out the database of sensor data described above. Furthermore, Blocks of the method 100 can be repeated over time for an individual user and/or multiple users, in order to generate models that describe longitudinal behavior of an alcohol sensing device sensor over time, as described in relation to the machine learning models and training data described above.

Furthermore, in relation to repetition of blocks of the method 100, each instance of biological sample provision can be performed without re-establishment of a baseline test result. For instance, if there is residual alcohol interacting with a sensor in association with biological sample provision and analysis, Blocks of the method 100 can be used to account for residual alcohol effects, such that the individual does not have to wait for an extended period of time between subsequent instances of biological sample provision.

The method 100 can, however, include any other suitable blocks or steps configured to facilitate use of data to provide more dynamic and accurate information related to alcohol sensing device test results. Furthermore, as a person skilled in the art will recognize from the previous detailed description and from the figures and claims, modifications and changes can be made to the method 100 without departing from the scope of the method 100.

### 2. System

As shown in FIG. 3A and 3B, an embodiment of a system 200 for characterization of a sensor state of an alcohol sensing device of an individual includes: a mobile computing device 220 of the individual, the mobile computing device; an alcohol sensing device 230 in communication with the mobile computing device 220 and configured to generate an alcohol signal upon reception of the biological sample; and a computing system 240 in communication with at least one of the mobile computing device 220 and the alcohol sensing device that functions to facilitate one or more of: receiving a supplementary dataset indicative of environmental metrics (e.g., conditions) associated with the alcohol sensing device sensor proximal in time to the time point of biological sample provision; receiving identifying information, including an age of the alcohol sensing device; based upon the supplementary dataset and the identifying information, correlating a profile of the alcohol signal to an correction factor of the alcohol sensing device sensor; and performing a correction operation configured to improve operation of the alcohol sensing device, based upon the correction factor.

In variations, the system 200 can be configured to perform at least a portion of the method 100 described in Section 1 above, and can additionally or alternatively be configured to perform any suitable method that increases accuracy or relevance of biological sample test information provided to individuals using alcohol sensing devices.

The system 200 can include elements as described in Section 1 above. The system 200 can additionally or alternatively include one or more embodiments, variations, and examples of system elements (e.g., biological sample acquisition device components, mobile computing device components, computing system components, etc.) described in U.S. application Ser. No. 14/169,029 entitled “Method and System for Monitoring Intoxication” and filed on 30-Jan.-2014, U.S. application Ser. No. 14/602,909 entitled “Method and System for Remotely Monitoring Intoxication” and filed on 22-Jan.-2015, U.S. application Ser. No. 14/631,125 entitled “Method and System for Monitoring Intoxication” and filed on 25-Feb.-2015, and U.S. application Ser. No. 15/375,801 entitled “Wearable System and Method for Monitoring Intoxication” and filed on 12-Dec.-2016, each of which is incorporated herein in its entirety by this reference. Variations of the system 100 can, however, be implemented at least in part using any other suitable system elements.

In variations of the system 200, the alcohol sensing device can include one or more hardware components or hardware modifications configured to provide more accurate sensor data, based upon environmental factors, user behaviors and/or other factors. For example, the system 200 can include a dynamically adjustable-gain amplifier, which can be used to selectively amplify portions of the response curve of the alcohol signal.

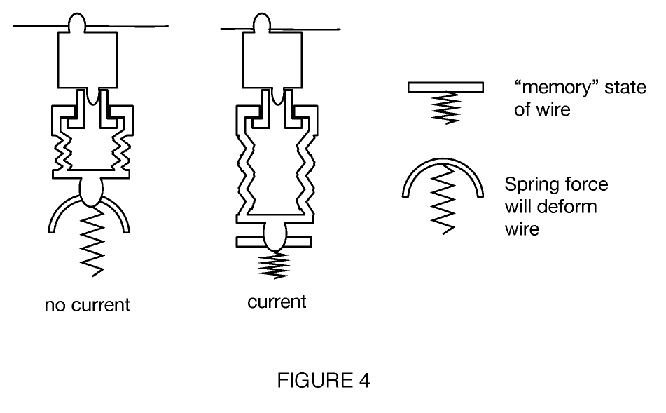

In one such variation, the system 200 can include components configured to provide improved airflow through the alcohol sensing device. In one variation, the system 200 can omit a solenoid (or otherwise include a minimized solenoid), wherein a wire and a spring (or other elastically deformable element) are coupled to the bellows. Additionally or alternatively, in some variations, as shown in FIG. 4, the system 200 can implement one or more shape memory elements (e.g., Nitinol wire, Flexinol wire, shape memory polymer component, etc.) that respond in desired ways to application of a current. In a specific example, application of a current within the sensor system can cause bellows of the system 200 to open by way of the shape memory element(s), in coordination with reception and processing of a biological sample to generate an alcohol signal. In another variation, actuators (e.g., servo motors, motors coupled to screw drives, etc.) coupled to the bellows can be implemented in the system 200 to transition the bellows between open and/or closed states. Additionally or alternatively, in another variation, electromagnetic components can interact with magnetic components coupled to the bellows in a manner that allows the bellows to transition between open and/or closed states. Such subsystems can be used to vary (e.g., meter) the amount of air intake appropriately.

Additionally or alternatively, in other variations, the system 200 can implement one or more pressure sensors that function to provide pressure data that can be used to increase the accuracy of alcohol sensing device sensor test outputs. In relation to multiple pressure sensors, the system 200 can thus account for sample provision specific effects related to inhalation (e.g., sucking) and exhalation (e.g., blowing), such as determining that a received breath sample is provided at the end of a breath action, and/or any other user behavior aspects.

Additionally or alternatively, in other variations, the system 200 can comprise a heating unit configured to heat one or more of the biological sample flow path and the alcohol sensing device sensor, in order to reduce condensation and/or mitigate environmental effects. In relation to heating, electronic components that generate heat during operation can be positioned proximal the sensor of the system 200, in order to utilize heat generated to heat one or more of the alcohol sensing device sensor and the flow path.

Additionally or alternatively, variations of the system 200 can implement removable elements/modules. For instance, one or more of the sensor and flow paths can be removable in order to provide efficient replacement solutions informed by analyses of sensor state or performance in coordination with Block S160 of the method 100 described above.

Additionally or alternatively, variations of the system 200 can modulate solenoid behavior (e.g., push-pull behavior) based upon environmental factors, sensor wear factors, and/or any other suitable factors. The system 200 can be configured to modulate solenoid behavior in firmware and/or in hardware mechanisms.

Additionally or alternatively, variations of the system 200 can include a module that dynamically adjusts the gain of signal amplification based upon anticipated sensor responses (e.g., in relation to specific values of an intoxication metric). For instance, the system 200 could be configured to dynamically adjust the gain of signal amplification in certain ranges of BAC readings in order to provide a higher degree of accuracy or precision in test outputs.

Additionally or alternatively, variations of the system 200 can be configured such that the user can select between receiving or determining information that is optimized for speed and information that is optimized for accuracy. For instance, information optimized for speed can be based upon analyses of a peak of an alcohol signal, while information that is optimized for accuracy can be based upon analysis of the integrated area of an alcohol signal. Alternatively, the system 200 can be configured to automatically switch between different modes (e.g., speed mode, accuracy mode, peak analysis mode, integrated area analysis mode) based upon detected environmental metrics, received supplementary datasets, extracted correction factors and/or any other suitable factors.

Additionally or alternatively, variations of the system 200 can interact with beacon systems (e.g., iBeacon, Estimote systems, etc.) in order to perform background functions, even when mobile applications associated with the system 100 are in an inactive state. In one example, the system 100 can be configured to enable background operations of the system 100 in line with the method 100 described above, whenever the system 100 interacts with a beacon system (e.g., if an iOS of an Apple device detects an iBeacon system). In an example operation of such a system 200, upon detection of a beacon system (e.g., using a BlueTooth LE advertising packet that facilitates invoking of devices when specific beacon types are detected), an individual can be prompted to turn on the alcohol sensing device and/or select a notification on a mobile application associated with the alcohol sensing device. Then, the biological sample provision process could be initiated within the application in a streamlined process. The beacon system can be associated with a specific environment.

Variations of the method 100 and system 200 include any combination or permutation of the described components and processes. Furthermore, various processes of the preferred method can be embodied and/or implemented at least in part as a machine configured to receive a computer-readable medium storing computer-readable instructions. The instructions are preferably executed by computer-executable components preferably integrated with a system and one or more portions of the control module 155 and/or a processor. The computer-readable medium can be implemented in the cloud and/or stored on any suitable computer readable media such as RAMs, ROMs, flash memory, EEPROMs, optical devices (CD or DVD), hard drives, floppy drives, or any suitable device. The computer-executable component is preferably a general or application specific processor, but any suitable dedicated hardware device or hardware/firmware combination device can additionally or alternatively execute the instructions.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, step, or portion of code, which includes one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

We claim:

1. A method for characterizing the state of an alcohol sensor comprising:
at a mobile computing device in communication with a fuel-cell alcohol sensing device, prompting an individual to provide a biological sample at a first time point;
at the fuel-cell alcohol sensing device, generating an alcohol signal upon reception of the biological sample from the individual;
at the fuel-cell alcohol sensing device, receiving an environmental metric associated with the fuel-cell alcohol sensing device at a second time point contemporaneous with the first time point;
at a remote computing system in communication with the mobile computing device, determining a degeneration parameter of the fuel-cell alcohol sensing device;
at the remote computing system, extracting a correction factor upon implementing a rule with the environmental metric and the degeneration parameter; and
at the remote computing system, generating a notification based upon the correction factor exceeding a threshold correction factor.

2. The method of claim 1, further comprising delivering the notification at the mobile computing device.

3. The method of claim 2, wherein the notification comprises an indication that the fuel-cell alcohol sensing device requires a replacement sensor.

4. The method of claim 1, wherein the alcohol signal comprises a response curve, and wherein determining the degeneration parameter of the blood alcohol sensing device comprises measuring a shape of the response curve and correlating the shape with the degeneration parameter.

5. The method of claim 4, wherein the response curve comprises a zero alcohol content response curve, and wherein correlating the shape with the degeneration parameter comprises comparing the zero alcohol content response curve to an ideal zero alcohol content response curve.

6. The method of claim 1, wherein receiving the environmental metric comprises detecting a humidity level at a humidity sensor of the fuel-cell alcohol sensing device.

7. The method of claim 1, further comprising receiving the biological sample from the individual at a cavity of the fuel-cell alcohol sensing device, the cavity comprising an aperture, and wherein prompting the individual to provide the biological sample comprises prompting the individual to provide a breath sample at the aperture.

8. The method of claim 1, further comprising receiving the biological sample from the individual at a skin contact region of the fuel-cell alcohol sensing device, and wherein the biological sample comprises a transdermally excreted fluid sample.

9. The method of claim 1, wherein the environmental metric is within a first range, wherein the degeneration parameter is within a second range, wherein the first and second range cooperatively define a two-dimensional parameter space, and wherein extracting the correction factor upon implementing the rule comprises selecting the correction factor from the two-dimensional parameter space based on the environmental metric and the degeneration parameter.

10. A method for characterizing the state of an alcohol sensor comprising:
at an alcohol sensing device, generating an alcohol signal upon reception of a biological sample from an individual at a first time point;
at the alcohol sensing device, receiving a supplementary dataset indicative of environmental metrics associated with the alcohol sensing device at a second time point contemporaneous with the first time point;
at a computing system in communication with the alcohol sensing device, determining a degeneration parameter of the alcohol sensing device;
at the computing system, extracting a correction factor upon implementing a rule with the supplementary dataset and the degeneration parameter; and
at the computing system, correcting the alcohol signal based upon the correction factor.

11. The method of claim 10, wherein the supplementary dataset comprises an operational lifetime of a sensor of the alcohol sensing device.

12. The method of claim 10, wherein receiving the supplementary dataset comprises measuring a temperature at a temperature sensor of the fuel-cell alcohol sensing device, and measuring a humidity level at a humidity sensor of the fuel-cell alcohol sensing device, and wherein the supplementary dataset comprises the temperature and the humidity level.

13. The method of claim 10, wherein implementing the rule comprises evaluating an extraction function, wherein an output of the extraction function comprises the correction factor and wherein inputs of the extraction function comprise the degeneration parameter and features of the supplementary dataset.

14. The method of claim 10, wherein correcting the alcohol signal based upon the correction factor comprises evaluating a correction function, wherein an input of the correction function comprises the correction factor and an output of the function comprises a corrected alcohol signal.

15. The method of claim 14, wherein evaluating the correction function comprises scaling the alcohol signal by the correction factor.

16. The method of claim 14, further comprising rendering, at a display of the computing system, a blood alcohol metric computed based upon the corrected alcohol signal.

17. The method of claim 10, wherein the correction factor comprises a corrective response curve, and wherein correcting the alcohol signal based upon the correction factor comprises convolving the corrective response curve with the alcohol signal to generate a corrected alcohol signal.

18. The method of claim 10, wherein the alcohol sensing device is operable between a first mode that directs incoming samples toward a primary sensor and a second mode that directs incoming biological samples toward a secondary sensor, and wherein correcting the alcohol signal based upon the correction factor comprises:
processing the correction factor with a criterion;
transitioning the alcohol sensing device between the first mode and the second mode upon detection that the correction factor fails to satisfy the criterion; and
generating a second alcohol signal upon reception of a second biological sample at the second sensor.

19. The method of claim 10, further comprising generating command instructions based on the supplementary dataset, and transmitting the command instructions from the computing system to an environmental control system that adjusts the environmental metrics associated with the alcohol sensing device in response to the command instructions.

20. The method of claim 10, further comprising receiving the biological sample from the individual at a skin contact region of the alcohol sensing device, and wherein the biological sample comprises a transdermally excreted fluid sample.
